(19) United States
(12) Patent Application Publication
(10) Pub. No.: US 2014/0234173 A1
(43) Pub. Date: Aug. 21, 2014

(54) STERILIZATION APPARATUS

(71) Applicant: HANSHIN MEDICAL CO., LTD., Bupyeong-gu (KR)

(72) Inventor: Jung Yeul Kim, Bupyeong-gu (KR)

(73) Assignee: HANSHIN MEDICAL CO., LTD., Bupyeong-gu (KR)

(21) Appl. No.: 14/172,846

(22) Filed: Feb. 4, 2014

(30) Foreign Application Priority Data
Feb. 20, 2013 (KR) 10-2013-0017960
Feb. 21, 2013 (KR) 10-2013-0018692

(51) Int. Cl. A61L 2/26

(52) U.S. Cl. CPC A61L 2/26
USPC 422/106; 422/292; 422/105

(57) ABSTRACT

Disclosed is a sterilization apparatus including a sterilant vaporizer main body including at least two first cavities and a first channel connecting the first cavities to each other, a cover including an inlet through which a sterilant flows and mechanically fastened with the sterilant vaporizer main body, and a sterilant heater heating the sterilant vaporizer main body to vaporize the sterilant flowing through the inlet.

### RELATED APPLICATIONS

This application claims priority under 35 U.S.C. §119 to Korean Patent Application Number 10-2013-0017960 filed on Feb. 20, 2013, and Korean Patent Application Number 10-2013-0018692 filed on Feb. 21, 2013. The entire contents of the foregoing applications are explicitly incorporated herein by reference.

### BACKGROUND OF THE INVENTION

1. Field of the Invention

The present invention relates to a sterilant apparatus vaporizing a sterilant in a liquid state to reduce a particle size in a gas state.

2. Discussion of the Related Art

Various types of medical appliances are used after sterilized by a sterilization apparatus to be free from microorganisms present on a surface and an inner cavity thereof. The sterilization apparatus includes a sterilant vaporization apparatus vaporizing a sterilant in a liquid state, and a vacuum pump connected to an inside of a chamber.

In a known method of vaporizing a sterilant in a liquid state, after the sterilant in the liquid state is injected into a storage space at a time, the storage space is heated at a time to vaporize the sterilant in the liquid state.

In the method, it takes a long time to vaporize the sterilant in the liquid state, and a particle size is not reduced well. Accordingly, a sterilization effect of the sterilant is reduced.

Meanwhile, moisture may be generated due to various causes such as a chemical reaction of the sterilant and an operation of the vacuum pump when a matter that is not completely dried and is to be sterilized is put into a sterilant machine and sterilized. The generated moisture may be contained in oil of the vacuum pump to cause a negative effect such as a reduction in performance and life-span of the vacuum pump and a reduction in sterilization effect.

As described above, moisture may reduce performance of the sterilizer. Accordingly, a technology of improving performance of the sterilization apparatus by effectively removing moisture needs to be studied.

Examples of prior art documents include ‘Korean Patent No. 10-0792964, title of the invention: VAPORIZER OF VAPORIZATION SYSTEM, and applicant: Shimadzu Corp.’, and a sterilant vaporizer described in this document vaporizes a sprayed liquid material.

For reference, examples of a prior art document which does not relate to the sterilization apparatus but may be referred include ‘Korean Patent No. 10-0412600, title of the invention: REMOVING APPARATUS OF CONDENSATE WATER OF EXHAUST SYSTEM, and applicant: Hyundai Motor Company’.

### SUMMARY

The present invention has been made in an effort to provide a technology of improving sterilizing power by vaporizing a liquid sterilant by using a plurality of cavities to reduce a particle size of the liquid sterilant within a short time.

The present invention has been made in an effort to also provide a technology of continuously maintaining performance of a vacuum pump and increasing convenience of a user by easily removing moisture included in oil used in the vacuum pump of a sterilization apparatus and informing the user about an oil change message when oil needs to be changed.

An exemplary embodiment of the present invention provides a sterilization apparatus including a sterilant vaporizer main body including at least two first cavities and a first channel connecting the first cavities to each other. A cover includes an inlet through which a sterilant flows and is mechanically fastened with the sterilant vaporizer main body. A sterilant heater heats the sterilant vaporizer main body to vaporize the sterilant flowing through the inlet.

Depths of the first cavities may be different from each other.

The first cavities may be divided into a first region and a second region. The depths of the first cavities included in the first region may be larger than depths of the first cavities included in the second region.

The first region may include the first cavities positioned at an inlet side.

The depths of the first cavities included in the first region may be 4 mm. The depths of the first cavities included in the second region may be 2 mm.

The first channel may sequentially connect the first cavities, and may be formed to have a depth that is smaller than the depths of the first cavities.

The sterilant vaporizer main body may further include a second cavity having a storage capacity that is larger than a storage capacity of one first cavity and containing the sterilant flowing through the inlet, and a second channel connecting any one of the first cavities and the second cavity.

The second cavity may have an inlet depth that is smaller than a depth thereof at the second channel.

The cover may further include an outlet provided on an upper surface of the cover, through which the sterilant vaporized by the sterilant heater is discharged.

The sterilization apparatus may further include a temperature sensor sensing a temperature, and a control unit controlling the sterilant heater to maintain a set temperature based on the sensed temperature.

The sterilization apparatus may further include a first oil tank including a first oil inlet through which oil flows from a vacuum pump, a first oil outlet through which oil in the first oil tank is discharged to the vacuum pump, and a first condensate water outlet through which condensate water is discharged. An oil level detection sensor may detect a level of oil included in the vacuum pump. A first valve may be connected to the first condensate water outlet of the first oil tank and adjust an amount of condensate water discharged from the first oil tank. A first driving unit may open and close the first valve. A control unit may drive the first driving unit so that condensate water is discharged only in a set amount to open and close the first valve when the detected level of oil is larger than a firstly set level of oil.

The control unit may display an oil change message in which a secondly set level of oil has a value that is larger than the firstly set level of oil on a display unit, or transmits the oil change message to a terminal of a user when the detected level of oil is larger than the secondly set level of oil.

The sterilization apparatus may further include an oil circulating pump formed between the vacuum pump and the first oil tank and circulating oil between the vacuum pump and the first oil tank.

The sterilization apparatus may further include a first piping formed between the first oil inlet and a second oil outlet formed on a lower end of the vacuum pump, and a second piping formed between the first oil outlet and a second oil inlet formed on an upper end of the vacuum pump.

The first oil tank may further include an oil filter removing an impurity included in oil flowing through the first oil inlet.

The first oil inlet and the first oil outlet may be formed on an upper end of the first oil tank. The first condensate water outlet may be formed on a lower end of the first oil tank.

The sterilization apparatus may further include a condensate water vaporizer including a storage unit connected to the first valve and storing condensate water flowing through the first valve, a condensate water heater heating the storage unit to vaporize condensate water, and a second condensate water outlet through which vaporized condensate water is discharged.

The control unit may drive the condensate water heater to vaporize condensate water flowing into the condensate water vaporizer.

The condensate water vaporizer may further include a temperature measurement sensor detecting a temperature of the storage unit. The control unit may control the condensate water heater to maintain a temperature of the condensate water heater at a set temperature.

The condensate water vaporizer may include the second condensate water outlet formed on an upper cover, through which vaporized condensate water is discharged to the atmosphere. The control unit may block power to the condensate water heater when the detected level of oil is smaller than the firstly set level of oil. The first oil tank may be formed outside the vacuum pump.

According to the disclosed invention, it is possible to improve sterilizing power by vaporizing a liquid sterilant by using a plurality of cavities to reduce a particle size of the liquid sterilant within a short time.

According to the disclosed invention, it is possible to continuously maintain performance of a vacuum pump by separately providing an oil tank and the vacuum pump and circulating oil by using an oil pump to prevent condensate water to stay in the vacuum pump over a long period of time.

Further, it is possible to solve inconvenience about separate discharging of condensate water from the oil tank by a user by automatically discharging condensate water from the oil tank according to a level of oil of the vacuum pump.

Further, it is possible to solve inconvenience about separate discharging of condensate water by the user by removing condensate water by using a condensate water vaporizer.

Further, the user can easily recognize whether oil needs to be changed by displaying an oil change message of the vacuum pump on a display unit or transmitting the oil change message to a terminal of the user.

### BRIEF DESCRIPTION OF THE DRAWINGS

FIG. 1 is a block diagram showing a sterilant vaporization apparatus relating to an exemplary embodiment of the present invention.

FIG. 2 is a view showing a sterilant vaporizer main body relating to the exemplary embodiment of the present invention.

FIG. 3 is a cross-sectional view of the sterilant vaporizer main body of FIG. 2, which is taken based on A.

FIG. 4 is a cross-sectional view of the sterilant vaporizer main body of FIG. 2, which is taken based on B.

FIG. 5 is a block diagram showing an oil circulating apparatus for vacuum pumps provided with a condensate water removing apparatus relating to the exemplary embodiment of the present invention.

FIG. 6 is a view showing the oil circulating apparatus for vacuum pumps provided with the condensate water removing apparatus relating to the exemplary embodiment of the present invention.

FIG. 7 is a flow chart showing a method of driving the oil circulating apparatus for vacuum pumps provided with the condensate water removing apparatus relating to the exemplary embodiment of the present invention.

### DETAILED DESCRIPTION

Hereinafter, exemplary embodiments of the present invention will be described in detail with reference to the accompanying drawings.

A sterilization apparatus according to the present invention includes a sterilant vaporization apparatus and an oil circulating apparatus for vacuum pumps as will be described below. The sterilization apparatus may further include another appropriate constitution sterilizing medical appliances.

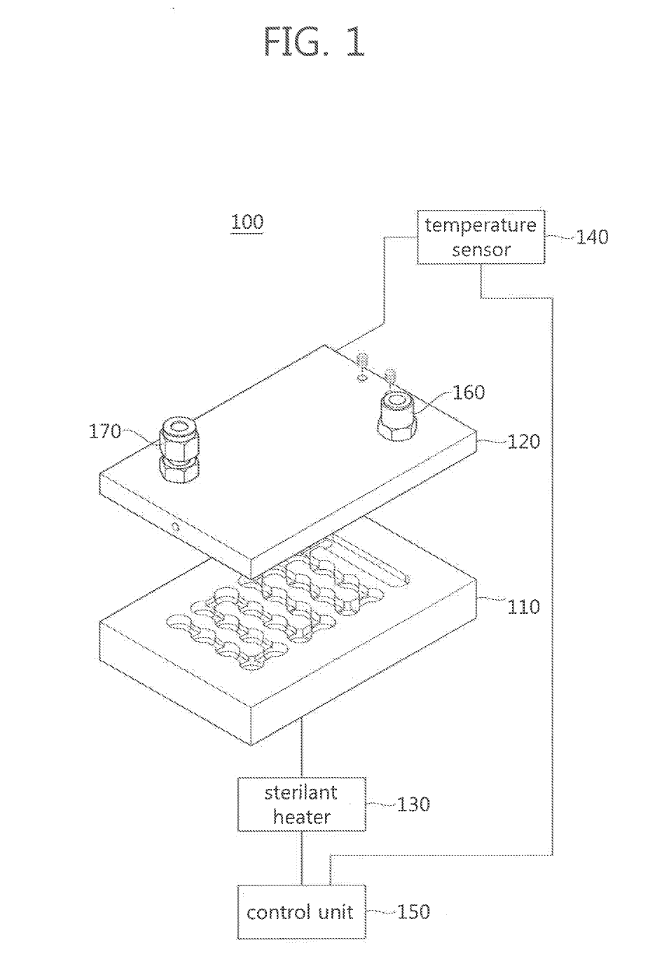

FIG. 1 is a block diagram showing a sterilant vaporization apparatus relating to an exemplary embodiment of the present invention.

Referring to FIG. 1, a sterilant vaporization apparatus 100 includes a sterilant vaporizer main body 110, a cover 120, a sterilant heater 130, a temperature sensor 140, and a control unit 150.

The sterilant vaporizer main body 110 may be provided with at least two first cavities, a first channel connecting the first cavities to each other, a second cavity containing a sterilant flowing through an inlet, and a second channel connecting the first cavities and the second cavity. The first cavities may have different depths. For example, a portion of the first cavities may have a depth of A mm, and the rest may have a depth of B mm. The first channel may sequentially connect the first cavities. The second cavity may contain the sterilant flowing through the inlet. The second channel may connect any one of the first cavities and the second cavity. The sterilant flowing through the inlet may move through the second channel to the first cavity.

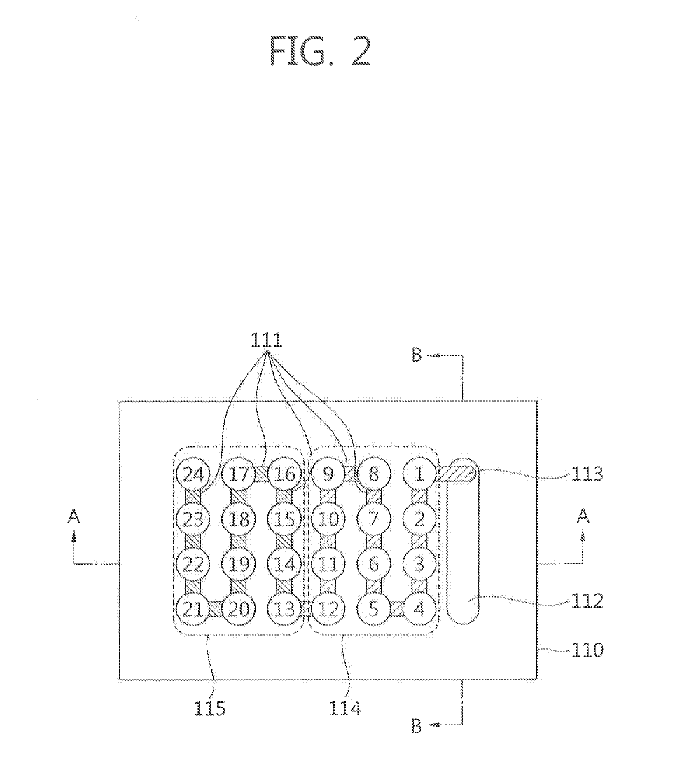

A specific description thereof will be given with reference to the following FIG. 2.

The cover 120 may be mechanically fastened with the sterilant vaporizer main body 110. For example, the cover 120 and the sterilant vaporizer main body 110 may be fastened by various methods such as fastening by various fastening means such as bolts/nuts, screws, and latches, and welding.

The cover 120 may be provided with an inlet 160 through which the sterilant flows and an outlet 170 through which the sterilant vaporized by the sterilant heater 130 is discharged. The inlet 160 and the outlet 170 may be provided on an upper surface of the cover 120.

The sterilant heater 130 may heat the sterilant vaporizer main body 110 to vaporize the sterilant flowing through the inlet 160. For example, the sterilant heater 130 may be provided inside or outside the sterilant vaporizer main body 110, or on an external lateral surface of the sterilant vaporizer main body 110. The sterilant heater 130 may be various types of heating apparatuses heating the sterilant vaporizer main body 110.

The temperature sensor 140 may sense a temperature. For example, the temperature sensor 140 may sense the temperature of the cover 120.

The control unit 150 may control the sterilant heater 130 so that a set temperature is maintained based on the sensed temperature. For example, when the set temperature is 200° C., the control unit 150 may operate or stop the sterilant heater 130 based on the sensed temperature to maintain the temperature at 200° C.

The sterilant vaporization apparatus may vaporize the liquid sterilant by using a plurality of cavities to reduce a particle size of the liquid sterilant within a short time, thus improving sterilizing power.

FIG. 2 is a view showing the sterilant vaporizer main body relating to the exemplary embodiment of the present invention.

Referring to FIG. 2, the sterilant vaporizer main body 110 may include first cavities 1 to 24, a first channel 111, a second cavity 112, and a second channel 113.

The number of first cavities 1 to 24 may be twenty four. In the present exemplary embodiment, the case where the number of cavities is twenty four is described as a standard. However, the number of cavities is not limited thereto but may be variously embodied.

The first cavities 1 to 24 may have different depths. For example, a portion of the first cavities may have a depth of A mm, and the rest may have a depth of B mm. The size of A mm may be larger than that of B mm. For example, the first cavities 1 to 24 may be divided into a first region 114 and a second region 115. The first region 114 may include the first cavities 1 to 12 positioned at an inlet side. The second region 115 may include the first cavities 13 to 24 positioned at an outlet side. Depths of the first cavities 1 to 12 included in the first region 114 may be larger than those of the first cavities 13 to 24 included in the second region 115. Specifically, for example, the depths of the first cavities 1 to 12 included in the first region 114 may be 4 mm. The depths of the first cavities 13 to 24 included in the second region 115 may be 2 mm.

The sterilant in a liquid state flows from the inlet 160 to the outlet 170, and the amount of the sterilant is gradually reduced while the sterilant flows toward the outlet 170.

Accordingly, the depths of the first cavities 1 to 12 included in the first region 114 may be set to be larger than those of the first cavities 13 to 24 included in the second region 115 to hold the sterilant in a more amount at the inlet 160 side at which the first cavities 1 to 12 included in the first region 114 are provided. Accordingly, vaporization performance of the sterilant in the liquid state may be improved.

In the present exemplary embodiment, for convenience of the description, the depths of the first cavities 1 to 12 and the depths of the first cavities 13 to 24 included in the second region 115 are limited, but numerical values are not limited thereto and may vary.

The first channel 111 may sequentially connect the first cavities 1 to 24.

In other words, the first channel 111 connects the first first cavity 1 and the second first cavity 2, the second first cavity 2 and the third first cavity 3, and the third first cavity 3 and the fourth first cavity 4. The first channel 111 may sequentially connect the first cavities 1 to 24 by the aforementioned connection method.

The first channel 111 may be formed to have a depth that is smaller than those of the first cavities 1 to 24.

The second cavity 112 may contain the sterilant flowing through the inlet. For example, the second cavity 112 may be formed to have a uniform depth. As another example, the second cavity 112 may be formed so that an inlet depth thereof is smaller than a depth thereof at the second channel 113. Accordingly, the sterilant flowing through the inlet may easily flow to the second channel 113.

A storage capacity of the second cavity 112 may be larger than the storage capacity of the one first cavity.

The second channel 113 may connect any one 1 of the first cavities 1 to 24 and the second cavity 112. The sterilant flowing through the inlet may move through the second channel 112 to the first cavity 1.

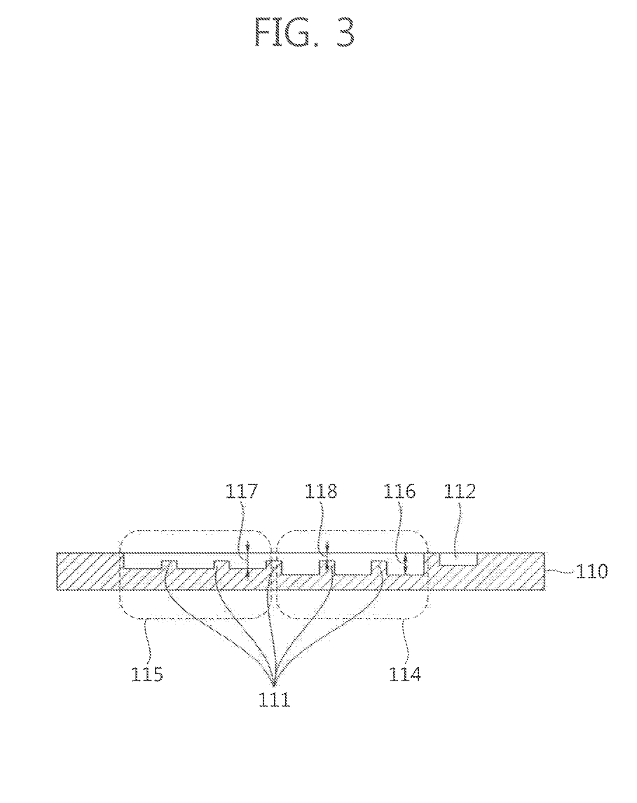

FIG. 3 is a cross-sectional view of the sterilant vaporizer main body of FIG. 2, which is taken based on A.

Referring to FIGS. 2 and 3, a depth 118 of the first channel 111 may be smaller than depths 116 of the first cavities 3, 6, and 11 included in the first region 114 and depths 117 of the first cavities 14, 19, and 22 included in the second region 115. Accordingly, the vaporized sterilant moves to a space between the channel 111 and the cover 120.

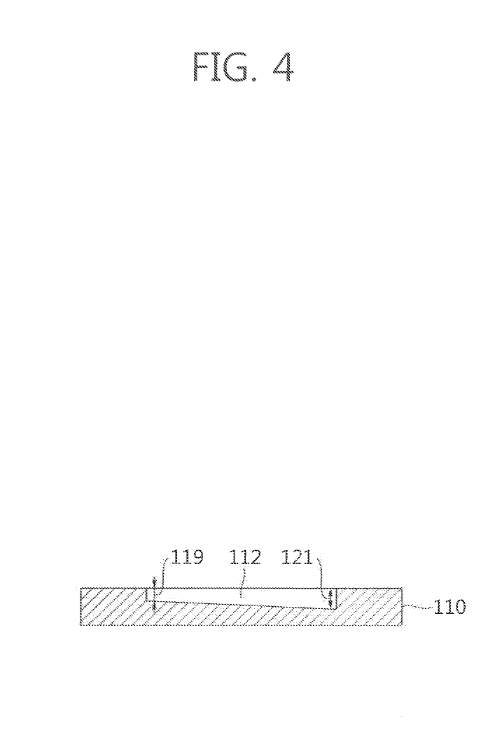

FIG. 4 is a cross-sectional view of the sterilant vaporizer main body of FIG. 2, which is taken based on B.

Referring to FIGS. 2 and 4, the second cavity 112 may contain the sterilant flowing through the inlet. For example, the second cavity 112 may be formed so that an inlet depth 119 thereof is smaller than a depth 121 thereof at the second channel 113. Accordingly, the sterilant flowing through the inlet may easily flow to the second channel 113.

Hereinafter, the oil circulating apparatus for vacuum pumps according to the present invention will be described.

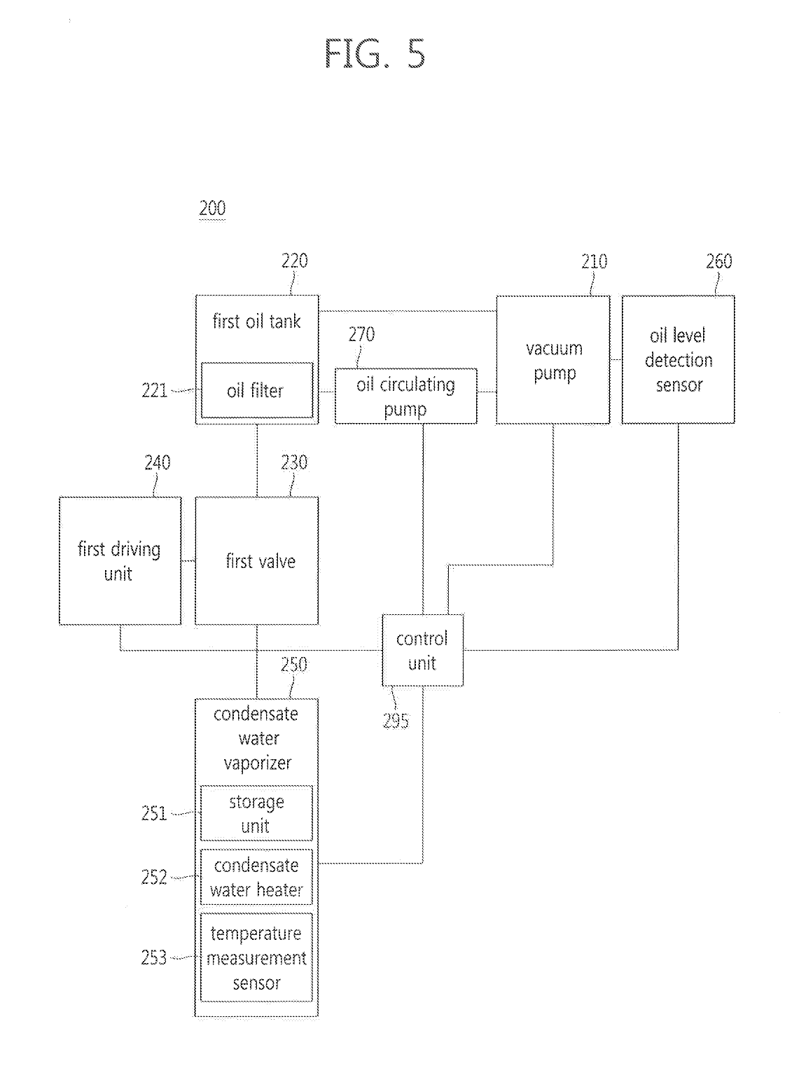

FIG. 5 is a block diagram showing the oil circulating apparatus for vacuum pumps provided with a condensate water removing apparatus relating to the exemplary embodiment of the present invention.

Referring to FIG. 5, an oil circulating apparatus for vacuum pumps 200 provided with the condensate water removing apparatus includes a vacuum pump 210, a first oil tank 220, a first valve 230, a first driving unit 240, a condensate water vaporizer 250, an oil level detection sensor 260, an oil circulating pump 270, and a control unit 295.

The first oil tank 220 may store oil usable in the vacuum pump 210. The first oil tank 220 may be formed outside the vacuum pump 210.

The first oil tank 220 may include a first oil inlet (not shown) through which oil flows from the vacuum pump 210, a first oil outlet (not shown) through which oil in the first oil tank 220 is discharged to the vacuum pump 210, and a first condensate water outlet (not shown) through which condensate water is discharged.

The first oil tank 220 may include an oil filter 221 removing an impurity included in oil flowing through the first oil inlet.

A first piping (not shown) may be formed between the first oil inlet and a second oil outlet formed on a lower end of the vacuum pump. A second piping may be formed between the first oil outlet and a second oil inlet formed on an upper end of the vacuum pump.

The first oil inlet and the first oil outlet of the first oil tank 220 may be formed on an upper end of the first oil tank 220. The condensate water outlet may be formed on a lower end of the first oil tank 220. Since condensate water sinks in the first oil tank 220, it is preferable that the condensate water outlet be formed at the lower end of the first oil tank 220.

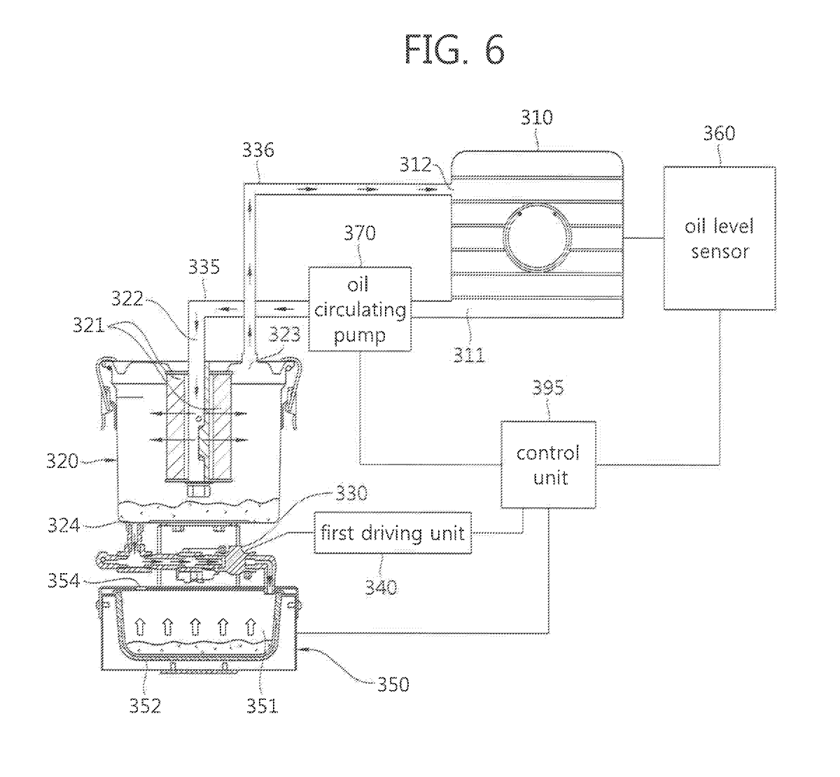

The first oil tank 220 will be specifically described with reference to the following FIG. 6.

The first valve 230 may be connected to the condensate water outlet (not shown) of the first oil tank 220, and adjust the amount of condensate water discharged from the first oil tank 220. For example, the first valve 230 may be various types of valves opened and closed like a solenoid valve.

The first driving unit 240 may open or close the first valve 230 according to a control signal of the control unit 295. For example, the first driving unit 240 may be various types of mechanical apparatuses opening or closing the valve.

The condensate water vaporizer 250 may be connected to the first valve 230. Specifically, an end of the first valve 230 is connected to the first oil tank 220, and another end of the first valve is connected to the condensate water vaporizer 250.

The condensate water vaporizer 250 includes a storage unit 251 storing condensate water flowing through the first valve 230, a condensate water heater 252 heating the storage unit 251 to vaporize condensate water, and a temperature measurement sensor 253 detecting the temperature of the condensate water vaporizer 250.

The condensate water heater 252 may be a heater having various structures or shapes heating the storage unit 251, for example, formed along an external surface of the storage unit 251 or formed while being spaced apart from the storage unit 251 at a predetermined interval.

The condensate water vaporizer 250 may include a second condensate water outlet (not shown) through which the vaporized condensate water is discharged. A specific description thereof will be given with reference to the following FIG. 6.

The oil level detection sensor 260 may detect a level of oil included in the vacuum pump 210. The oil level detection sensor 260 may transmit detected oil level information to the control unit 295.

The oil circulating pump 270 may be formed between the vacuum pump 210 and the first oil tank 220. The oil circulating pump 270 may circulate oil between the vacuum pump 210 and the first oil tank 220. Specifically, the oil circulating pump 270 may allow oil in the first oil tank 220 to flow into the vacuum pump 210 and oil in the vacuum pump 210 to flow into the first oil tank 220.

The control unit 295 may control constituent elements included in the present invention. For example, the control unit 295 may control the vacuum pump 210, the first driving unit 240, the condensate water vaporizer 250, and the oil circulating pump 270.

The control unit 295 may drive the first driving unit 240 to open and close the first valve 230 so that condensate water is discharged in a set amount to the condensate water vaporizer 250 when the level of oil detected by the oil level detection sensor 260 is larger than a firstly set level of oil. The firstly set level of oil may be set by a user or a manufacturer. For example, when the detected level of oil is larger than the firstly set level of oil, the control unit 295 may drive the first driving unit 240 to open and close the first valve 230 so that condensate water is discharged in an amount of 100 ml or less to the condensate water vaporizer 250.

When the detected level of oil is larger than a secondly set level of oil, the control unit 295 may display an oil change message on a display unit (not shown), or transmit the oil change message through a communication unit (not shown) to a terminal of the user. Herein, the secondly set level of oil may have a value that is larger than the firstly set level of oil. The secondly set level of oil is an oil level corresponding to the case where changing of oil is more preferable than removal of condensate water, and may be set by the user or the manufacturer.

The control unit 295 may drive the condensate water heater 252 to vaporize condensate water flowing into the condensate water vaporizer 250. For example, the control unit 295 may control the condensate water heater 252 to maintain a temperature of the condensate water heater 252 at a set temperature based on the temperature detected by the temperature measurement sensor 253.

As another example, the control unit 295 may block power to the condensate water heater 252 when the detected level of oil is smaller than the firstly set level of oil. On the contrary, the control unit 295 may apply power to the condensate water heater 252 when the detected level of oil is larger than the firstly set level of oil. As described above, power consumption may be significantly reduced by controlling power of the condensate water heater 252 according to a comparison result of the detected level of oil and the firstly set level of oil.

The oil circulating apparatus for vacuum pumps provided with the condensate water removing apparatus may be separately provided with the oil tank and the vacuum pump and circulate oil by using an oil pump. Accordingly, condensate water may be prevented from staying in the vacuum pump over a long period of time to continuously maintain performance of the vacuum pump.

Further, the oil circulating apparatus for vacuum pumps provided with the condensate water removing apparatus may solve inconvenience about separate discharging of condensate water from the oil tank by the user by automatically discharging condensate water from the oil tank according to the level of oil of the vacuum pump.

Further, the oil circulating apparatus for vacuum pumps provided with the condensate water removing apparatus may solve inconvenience about separate discharging of condensate water by the user by removing condensate water by using the condensate water vaporizer.

Further, the oil circulating apparatus for vacuum pumps provided with the condensate water removing apparatus may display the oil change message of the vacuum pump on the display unit or transmit the oil change message to the terminal of the user. Accordingly, the user may easily recognize whether oil needs to be changed.

FIG. 6 is a view showing the oil circulating apparatus for vacuum pumps provided with the condensate water removing apparatus relating to the exemplary embodiment of the present invention.

Referring to FIG. 6, an oil circulating apparatus for vacuum pumps 300 provided with the condensate water removing apparatus includes a vacuum pump 310, a first oil tank 320, a first valve 330, a first driving unit 340, a condensate water vaporizer 350, an oil level detection sensor 360, an oil circulating pump 370, and a control unit 395.

The first oil tank 320 may store oil usable in the vacuum pump 310. The first oil tank 320 may be formed outside the vacuum pump 310.

The first oil tank 320 may include a first oil inlet 322 through which oil flows from the vacuum pump 310, a first oil outlet 323 through which oil in the first oil tank 320 is discharged to the vacuum pump 310, and a first condensate water outlet 324 through which condensate water is discharged.

The first oil tank 320 may include an oil filter 321 removing an impurity included in oil flowing through the first oil inlet 322. The oil filter 321 may function to filter the impurity while oil flowing through the first oil inlet 322 passing through the oil filter 321. The oil filter 321 may be formed to surround a space receiving inflow oil, or may be integrally provided with the space receiving inflow oil. Further, a mechanical constitution blocking oil may be integrally or separately provided with the oil filter 321 on the lowermost surface of the oil filter 321 to pass inflow oil through only the oil filter 321. In other words, the first oil tank 320 may include various types of mechanical constitutions where inflow oil passes through only the oil filter 321.

A first piping 335 may be formed between the first oil inlet 322 and a second oil outlet 311 formed on a lower end of the vacuum pump 310.

A second piping 336 may be formed between the first oil outlet 323 and a second oil inlet 312 formed on an upper end of the vacuum pump 310.

The first oil inlet 322 and the first oil outlet 323 of the first oil tank 320 may be formed on an upper end of the first oil tank 320.

The condensate water outlet 324 may be formed on a lower end of the first oil tank 320.

Since condensate water sinks in the first oil tank 320, it is preferable that the condensate water outlet 324 be formed on the lower end of the first oil tank 320.

The first valve 330 may be connected to the condensate water outlet 324 of the first oil tank 320, and adjust the amount of condensate water discharged from the first oil tank 320. For example, the first valve 330 may directly connect the condensate water outlet 324 and the condensate water vaporizer 350. As another example, piping may be formed between the first valve 330 and the condensate water outlet 324, and between the first valve 330 and the condensate water vaporizer 350. The first valve 330 may be formed between the piping.

For example, the first valve 330 may be various types of valves opened and closed like a solenoid valve.

The first driving unit 340 may open or close the first valve 330 according to a control signal of the control unit 395. For example, the first driving unit 340 may be various types of mechanical apparatuses opening or closing the valve.

The condensate water vaporizer 350 may be connected to the first valve 330. Specifically, an end of the first valve is connected to the first oil tank 320, and another end of the first valve is connected to the condensate water vaporizer 350.

The condensate water vaporizer 350 may include a storage unit 351 storing condensate water flowing through the first valve 330, a condensate water heater 352 heating the storage unit to vaporize condensate water, and a second condensate water outlet 354 through which vaporized condensate water is removed.

The condensate water heater 352 may be a heater having various structures or shapes heating the storage unit 351, for example, formed along an external surface of the storage unit 351 or formed while being spaced apart from the storage unit 351 at a predetermined interval.

The second condensate water outlet 354 may be formed on an upper cover of the condensate water vaporizer 350. Vaporized condensate water may be discharged through the second condensate water outlet 354 to the atmosphere.

The oil level detection sensor 360 may detect a level of oil included in the vacuum pump 310. The oil level detection sensor 360 may transmit detected oil level information to the control unit 395.

The oil circulating pump 370 may be formed between the vacuum pump 310 and the first oil tank 320. The oil circulating pump 370 may circulate oil between the vacuum pump 310 and the first oil tank 320. Specifically, the oil circulating pump 370 may allow oil in the first oil tank 320 to flow into the vacuum pump 310 and oil in the vacuum pump 310 to flow into the first oil tank 320.

The control unit 395 may control constituent elements included in the present invention. For example, the control unit 395 may control the vacuum pump 310, the first driving unit 340, the condensate water vaporizer 350, and the oil circulating pump 370.

The control unit 395 may drive the first driving unit 340 to open and close the first valve 330 so that condensate water is discharged in a set amount to the condensate water vaporizer 350 when the level of oil detected by the oil level detection sensor 360 is larger than the firstly set level of oil. The firstly set level of oil may be set by the user or the manufacturer. For example, when the detected level of oil is larger than the firstly set level of oil, the control unit 395 may drive the first driving unit 340 to open and close the first valve 330 so that condensate water is discharged in an amount of 100 ml or less to the condensate water vaporizer 350.

When the detected level of oil is larger than the secondly set level of oil, the control unit 395 may display the oil change message on the display unit (not shown), or transmit the oil change message through the communication unit (not shown) to the terminal of the user. Herein, the secondly set level of oil may have a value that is larger than the firstly set level of oil. The secondly set level of oil is an oil level corresponding to the case where changing of oil is more preferable than removal of condensate water, and may be set by the manufacturer.

The control unit 395 may drive the condensate water heater 352 to vaporize condensate water flowing into the condensate water vaporizer 350. For example, the control unit 395 may control the condensate water heater 352 to maintain a temperature of the condensate water heater 352 at a set temperature based on the temperature detected by a temperature measurement sensor (not shown).

The oil circulating apparatus for vacuum pumps provided with the condensate water removing apparatus may be separately provided with the oil tank and the vacuum pump and circulate oil by using an oil pump. Accordingly, condensate water may be prevented from staying in the vacuum pump over a long period of time to continuously maintain performance of the vacuum pump.

Further, the oil circulating apparatus for vacuum pumps provided with the condensate water removing apparatus may solve inconvenience about separate discharging of condensate water from the oil tank by the user by automatically discharging condensate water from the oil tank according to the level of oil of the vacuum pump.

Further, the oil circulating apparatus for vacuum pumps provided with the condensate water removing apparatus may solve inconvenience about separate discharging of condensate water by the user by removing condensate water by using the condensate water vaporizer.

Further, the oil circulating apparatus for vacuum pumps provided with the condensate water removing apparatus may display the oil change message of the vacuum pump on the display unit or transmit the oil change message to the terminal of the user. Accordingly, the user may easily recognize whether oil needs to be changed.

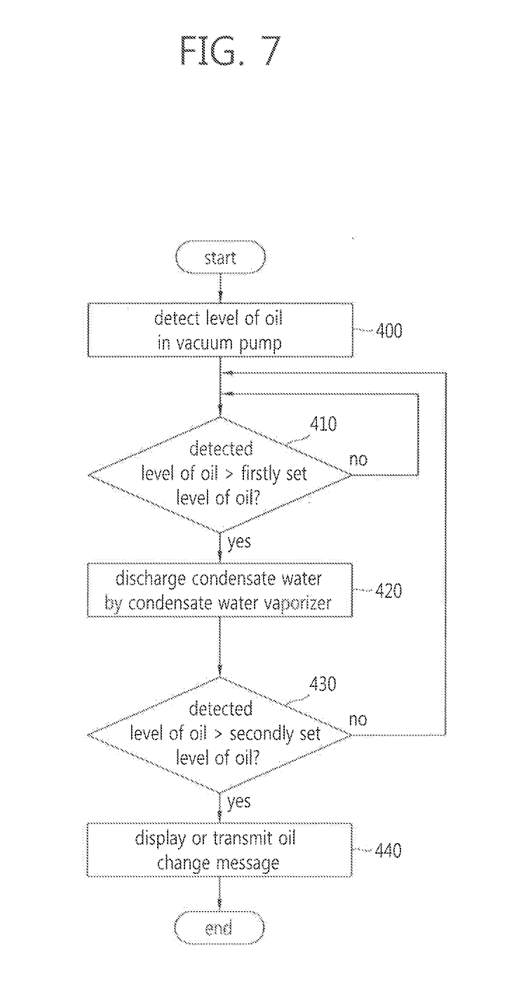

FIG. 7 is a flow chart showing a method of driving the oil circulating apparatus for vacuum pumps provided with the condensate water removing apparatus relating to the exemplary embodiment of the present invention.

Referring to FIGS. 5 and 7, the oil circulating apparatus for vacuum pumps 200 provided with the condensate water removing apparatus detects the level of oil in the vacuum pump 400. The oil circulating apparatus for vacuum pumps 200 provided with the condensate water removing apparatus judges whether the detected level of oil is larger than the firstly set level of oil 410. When the detected level of oil is smaller than the firstly set level of oil, the oil circulating apparatus for vacuum pumps 200 provided with the condensate water removing apparatus performs step 410 again. On the other hand, when the detected level of oil is larger than the firstly set level of oil, the oil circulating apparatus for vacuum pumps 200 provided with the condensate water removing apparatus drives the first driving unit to open and close the first valve. Thereby, condensate water is discharged in a set amount to the condensate water vaporizer 420.

The oil circulating apparatus for vacuum pumps 200 provided with the condensate water removing apparatus judges whether the detected level of oil is larger than the secondly set level of oil 430. When the detected level of oil is smaller than the secondly set level of oil, the oil circulating apparatus for vacuum pumps 200 provided with the condensate water removing apparatus performs step 410 again. On the other hand, when the detected level of oil is larger than the secondly set level of oil, the oil circulating apparatus for vacuum pumps 200 provided with the condensate water removing apparatus displays the oil change message on the display unit (not shown), or transmits the oil change message through the communication unit (not shown) to the terminal of the user 440.

As another example, the oil circulating apparatus for vacuum pumps 200 provided with the condensate water removing apparatus may drive the condensate water heater to vaporize condensate water flowing into the condensate water vaporizer.

All or a portion of the exemplary embodiments may be selectively combined in order to perform various modifications.

Further, it should be noted that it the exemplary embodiments are set forth to illustrate, but are not to be construed to limit the present invention. Further, it can be understood that numerous other modifications and embodiments can be devised by those skilled in the art within the scope of the present invention.

What is claimed is:

1. A sterilization apparatus comprising:
a sterilant vaporizer main body including at least two first cavities and a first channel connecting the first cavities to each other,
a cover including an inlet through which a sterilant flows and mechanically fastened with the sterilant vaporizer main body, and
a sterilant heater heating the sterilant vaporizer main body to vaporize the sterilant flowing through the inlet.

2. The sterilization apparatus of claim 1, wherein depths of the first cavities are different from each other.

3. The sterilization apparatus of claim 1, wherein the first cavities are divided into a first region and a second region, and depths of the first cavities included in the first region are larger than depths of the first cavities included in the second region.

4. The sterilization apparatus of claim 3, wherein the first region includes the first cavities positioned at an inlet side.

5. The sterilization apparatus of claim 3, wherein the depths of the first cavities included in the first region are 4 mm, and the depths of the first cavities included in the second region are 2 mm.

6. The sterilization apparatus of claim 1, wherein the first channel sequentially connects the first cavities, and is formed to have a depth that is smaller than depths of the first cavities.

7. The sterilization apparatus of claim 1, wherein the sterilant vaporizer main body further includes
a second cavity having a storage capacity that is larger than a storage capacity of one first cavity and containing the sterilant flowing through the inlet, and
a second channel connecting any one of the first cavities and the second cavity.

8. The sterilization apparatus of claim 7, wherein the second cavity has an inlet depth that is smaller than a depth thereof at the second channel.

9. The sterilization apparatus of claim 1, wherein the cover further includes an outlet provided on an upper surface of the cover, through which the sterilant vaporized by the sterilant heater is discharged.

10. The sterilization apparatus of claim 1, further comprising:
a temperature sensor sensing a temperature, and
a control unit controlling the sterilant heater to maintain a set temperature based on a sensed temperature.

11. The sterilization apparatus of claim 1, further comprising:
a first oil tank including a first oil inlet through which oil flows from a vacuum pump, a first oil outlet through which the oil in the first oil tank is discharged to the vacuum pump, and a first condensate water outlet through which condensate water is discharged,
an oil level detection sensor detecting a level of the oil included in the vacuum pump,
a first valve connected to the first condensate water outlet of the first oil tank and adjusting an amount of the condensate water discharged from the first oil tank,
a first driving unit opening and closing the first valve, and
a control unit driving the first driving unit so that the condensate water is discharged only in a set amount to open and close the first valve when a detected level of the oil is larger than a firstly set level of the oil.

12. The sterilization apparatus of claim 11, wherein the control unit displays an oil change message in which a secondly set level of the oil has a value that is larger than the firstly set level of the oil on a display unit, or transmits the oil change message to a terminal of a user when the detected level of the oil is larger than the secondly set level of the oil.

13. The sterilization apparatus of claim 11, further comprising:
an oil circulating pump formed between the vacuum pump and the first oil tank and circulating the oil between the vacuum pump and the first oil tank.

14. The sterilization apparatus of claim 11, further comprising:
a first piping formed between the first oil inlet and a second oil outlet formed on a lower end of the vacuum pump, and
a second piping formed between the first oil outlet and a second oil inlet formed on an upper end of the vacuum pump.

15. The sterilization apparatus of claim 11, wherein the first oil tank further includes an oil filter removing an impurity included in the oil flowing through the first oil inlet.

16. The sterilization apparatus of claim 11, wherein the first oil inlet and the first oil outlet are formed on an upper end of the first oil tank, and the first condensate water outlet is formed on a lower end of the first oil tank.

17. The sterilization apparatus of claim 11, further comprising:
a condensate water vaporizer including a storage unit connected to the first valve and storing the condensate water flowing through the first valve, a condensate water heater heating the storage unit to vaporize the condensate water, and a second condensate water outlet through which vaporized condensate water is discharged.

18. The sterilization apparatus of claim 17, wherein a control unit drives the condensate water heater to vaporize the condensate water flowing into the condensate water vaporizer.

19. The sterilization apparatus of claim 17, wherein the condensate water vaporizer further includes a temperature measurement sensor detecting a temperature of the storage unit, and a control unit controls the condensate water heater to maintain a temperature of the condensate water heater at a set temperature.

20. The sterilization apparatus of claim 17, wherein the condensate water vaporizer includes the second condensate water outlet formed on an upper cover, through which the vaporized condensate water is discharged to an atmosphere, a control unit cuts off power to the condensate water heater when a detected level of oil is smaller than a firstly set level of the oil, and a first oil tank is formed outside a vacuum pump.
